(19) United States
(12) Patent Application Publication
(10) Pub. No.: US 2025/0120957 A1
(43) Pub. Date: Apr. 17, 2025

(54) TREATMENT OF NEUROPSYCHIATRIC DISORDERS WITH TILIVAPRAM

(71) Applicant: Alto Neuroscience, Inc., Mountain View, CA (US)

(72) Inventors: Amit ETKIN, Mountain View, CA (US); Adam SAVITZ, Mountain View, CA (US); Wei WU, Mountain View, CA (US); Joshua JORDAN, Mountain View, CA (US); Akshay SUJATHA RAVINDRAN, Mountain View, CA (US); Bruce MORIMOTO, Mountain View, CA (US); Chao WANG, Mountain View, CA (US); Guhan SUNDAR, Mountain View, CA (US)

(21) Appl. No.: 18/896,294

(22) Filed: Sep. 25, 2024

Related U.S. Application Data
(60) Provisional application No. 63/668,377, filed on Jul. 8, 2024.
(60) Provisional application No. 63/650,360, filed on May 21, 2024.
(60) Provisional application No. 63/634,306, filed on Apr. 15, 2024.
(60) Provisional application No. 63/589,729, filed on Oct. 12, 2023.

(51) Int. Cl. A61K 31/444; A61K 31/4418; A61K 45/06; A61P 25/28

(52) U.S. Cl. CPC A61K 31/444; A61K 31/4418; A61K 45/06; A61P 25/28

(57) ABSTRACT

This invention relates to the treatment of a neuropsychiatric disorder, such as schizophrenia or Parkinson's disease, by administration (for example, transdermally) of tilivapram, zatolmilast, roflumilast, or a pharmaceutically acceptable salt thereof.

This application claims the benefit of U.S. Patent Application No. 63/589,729, filed Oct. 12, 2023, U.S. Patent Application No. 63/634,306, filed Apr. 15, 2024, U.S. Patent Application No. 63/650,360, filed May 21, 2024, and U.S. Patent Application No. 63/668,377, filed Jul. 8, 2024, each of which is hereby incorporated by reference.

### FIELD OF THE INVENTION

The present invention relates to the treatment of a neuropsychiatric disorder, such as schizophrenia or Parkinson's disease, by administration (for example, transdermally) of tilivapram, zatolmilast, roflumilast, or a pharmaceutically acceptable salt thereof.

### BACKGROUND OF THE INVENTION

Tilivapram (4-(cyclopropylmethoxy)-N-(3,5-dichloro-1-oxidopyridin-4-yl)-5-methoxypyridine-2-carboxamide), which is described in U.S. Pat. No. 9,611,250 and International Publication No. WO 95/04045, is a phosphodiesterase 4 (PDE4) inhibitor.

There is a continuing need for improved treatments for neuropsychiatric disorders such as schizophrenia and Parkinson's disease.

### SUMMARY OF THE INVENTION

One embodiment is a method of treating a neuropsychiatric disorder in a patient in need thereof comprising administering (for instance, orally or transdermally) to the patient an effective amount of tilivapram, zatolmilast (BPN14770), roflumilast, or a pharmaceutically acceptable salt thereof. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch.

Another embodiment is a method of treating a neuropsychiatric disorder in a patient in need thereof comprising administering (for instance, orally or transdermally) to the patient an effective amount of (a) tilivapram, zatolmilast, roflumilast, or a pharmaceutically acceptable salt thereof and (b) one or more antipsychotic agents. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch.

In one embodiment, the neuropsychiatric disorder is Parkinson's disease. In another embodiment, the neuropsychiatric disorder is cognitive impairment associated with Parkinson's disease. In yet another embodiment, the neuropsychiatric disorder is memory impairment associated with Parkinson's disease. In yet another embodiment, the neuropsychiatric disorder is information processing speed and/or psychomotor speed impairment associated with Parkinson's disease.

In another embodiment, the neuropsychiatric disorder is schizophrenia. In another embodiment, the neuropsychiatric disorder is cognitive impairment associated with schizophrenia. In yet another embodiment, the neuropsychiatric disorder is memory impairment associated with schizophrenia. In yet another embodiment, the neuropsychiatric disorder is information processing speed and/or psychomotor speed impairment associated with schizophrenia.

In yet another embodiment, the neuropsychiatric disorder is mild cognitive impairment (MCI).

In yet another embodiment, the neuropsychiatric disorder is attention deficit hyperactivity disorder (ADHD).

Other neuropsychiatric disorders which can be treated with tilivapram (e.g., transdermal tilivapram), zatolmilast (BPN14770), roflumilast, or a pharmaceutically acceptable salt thereof include, but are not limited to, conditions associated with cerebral metabolic inhibition, depression, bipolar disorder, post-traumatic stress disorder (PTSD), autism, Fragile X disorder, Angelman's syndrome, cognitive impairment, Alzheimer's disease (AD), frontotemporal dementia, vascular dementia, dementia with Lewy bodies (DLB), migraine, developmental delay, learning disabilities, multiple sclerosis, and amyotrophic lateral sclerosis (ALS).

In one embodiment of any of the methods described herein, the patient suffers from Parkinson's disease.

In one embodiment of any of the methods described herein, the patient suffers from cognitive impairment associated with Parkinson's disease.

In one embodiment of any of the methods described herein, the patient suffers from memory impairment associated with Parkinson's disease.

In one embodiment of any of the methods described herein, the patient suffers from information processing and/or psychomotor speed impairment associated with Parkinson's disease.

In one embodiment of any of the methods described herein, the patient suffers from schizophrenia.

In one embodiment of any of the methods described herein, the patient suffers from cognitive impairment associated with schizophrenia.

In one embodiment of any of the methods described herein, the patient suffers from memory impairment associated with schizophrenia.

In one embodiment of any of the methods described herein, the patient suffers from information processing and/or psychomotor speed impairment associated with schizophrenia.

In one embodiment of any of the methods described herein, the patient suffers from mild cognitive impairment (MCI).

In one embodiment of any of the methods described herein, the patient suffers from attention deficit hyperactivity disorder (ADHD).

In one embodiment of any of the methods described herein, the patient has impaired cognition.

In one embodiment of any of the methods described herein, the patient has impaired memory.

In one embodiment of any of the methods described herein, the one or more antipsychotic agents are selected from olanzapine, risperidone, quetiapine, sertindole, amisulpride, aripiprazole, asenapine, blonanserin, bifeprunox, cariprazine, clotiapine, iloperidone, lurasidone, mosapramine, melperone, paliperidone, perospirone, pimavanserin, remoxipride, sulpiride, ziprasidone, zotepine, perphenazine, thioridazine, chlorpromazine, brexpiprazole, lumateperone (e.g., lumateperone tosylate), fluphenazine, haloperidol, loxapine, prochlorperazine, flupentixol, levomepromazine, periciazine, pimozide, promazine, trifluperazine, zuclopenthixol, molindone, thiothixene, thoridazine, trifluoperazine, pharmaceutically acceptable salts thereof, and any combination of any of the foregoing. In another embodiment, the antipsychotic agent is quetiapine, lurasidone, cariprazine, or a combination of olanzapine and fluoxetine. In yet another embodiment, the antipsychotic agent is a combination of brexpiprazole and sertraline.

Another embodiment is a method of treating negative symptoms of schizophrenia in a patient in need thereof comprising administering (for instance, orally or transdermally) to the patient an effective amount of tilivapram, zatolmilast (BPN14770), roflumilast, or a pharmaceutically acceptable salt thereof. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch. Negative symptoms can include blunted affect, alogia (reduction in quantity of words spoken), avolition (reduced goal-directed activity due to decreased motivation), asociality, anhedonia (reduced experience of pleasure), or any combination of any of the foregoing.

Yet another embodiment is a method of decreasing theta power on a resting state electroencephalogram (EEG), increasing theta intertrial coherence, increasing gamma-band phase locking in response to stimulus registration as measured by EEG, increasing mismatch negativity in an oddball task as measured by EEG, increasing processing speed, increasing psychomotor speed or any combination of any of the foregoing in a patient, such as a patient suffering from a neuropsychiatric disorder, comprising administering (for instance, orally or transdermally) to the patient an effective amount of tilivapram, zatolmilast (BPN14770), roflumilast, or a pharmaceutically acceptable salt thereof. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch.

Yet another embodiment is a method of decreasing theta power on a resting state electroencephalogram (EEG), increasing theta intertrial coherence, increasing gamma-band phase locking in response to stimulus registration as measured by EEG, increasing mismatch negativity in an oddball task as measured by EEG, increasing processing speed, increasing psychomotor speed or any combination of any of the foregoing in a patient, such as a patient suffering from a neuropsychiatric disorder, comprising administering (for instance, orally or transdermally) to the patient an effective amount of (a) tilivapram, zatolmilast (BPN14770), roflumilast, or a pharmaceutically acceptable salt thereof and (b) one or more antipsychotic agents. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch.

Yet another embodiment is a method of improving cognition in a patient in need thereof (for example, a patient having a neuropsychiatric disorder) comprising administering (for instance, orally or transdermally) to the patient an effective amount of tilivapram, zatolmilast (BPN14770), roflumilast, or a pharmaceutically acceptable salt thereof. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch.

Yet another embodiment is a method of improving cognition in a patient in need thereof (for example, a patient having a neuropsychiatric disorder) comprising administering (for instance, orally or transdermally) to the patient an effective amount of (a) tilivapram, zatolmilast (BPN14770), roflumilast, or a pharmaceutically acceptable salt thereof and (b) one or more antipsychotic agents. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch.

Yet another embodiment is a method of improving memory in a patient in need thereof (for example, a patient having a neuropsychiatric disorder) comprising administering (for instance, orally or transdermally) to the patient an effective amount of tilivapram, zatolmilast (BPN14770), roflumilast, or a pharmaceutically acceptable salt thereof. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch.

Yet another embodiment is a method of improving memory in a patient in need thereof (for example, a patient having a neuropsychiatric disorder) comprising administering (for instance, orally or transdermally) to the patient an effective amount of (a) tilivapram, zatolmilast (BPN14770), roflumilast, or a pharmaceutically acceptable salt thereof and (b) one or more antipsychotic agents. In one embodiment, the tilivapram or a pharmaceutically acceptable salt thereof is transdermally administered to the patient. In one embodiment, the tilivapram free base is transdermally administered to the patient. In another embodiment, the tilivapram or a pharmaceutically acceptable salt thereof is administered via a transdermal patch.

In one embodiment of any of the methods described herein, prior to initiating treatment with tilivapram, zatolmilast, roflumilast, or a pharmaceutically acceptable salt thereof, the patient is treated with the one or more antipsychotic agents but did not (i) adequately respond to the antipsychotic agents to treat the neuropsychiatric disorder, or (ii) achieve the desired control of the neuropsychiatric disorder.

In one embodiment of any of the methods described herein, the one or more antipsychotic agents are selected from olanzapine, risperidone, quetiapine, sertindole, amisulpride, aripiprazole, asenapine, blonanserin, bifeprunox, cariprazine, clotiapine, iloperidone, lurasidone, mosapramine, melperone, paliperidone, perospirone, pimavanserin, remoxipride, sulpiride, ziprasidone, zotepine, perphenazine, thioridazine, chlorpromazine, brexpiprazole, lumateperone (e.g., lumateperone tosylate), fluphenazine, haloperidol, loxapine, prochlorperazine, flupentixol, levomepromazine, periciazine, pimozide, promazine, trifluperazine, zuclopenthixol, molindone, thiothixene, thoridazine, trifluoperazine, pharmaceutically acceptable salts thereof, or any combination of any of the foregoing. In another embodiment, the antipsychotic agent is quetiapine, lurasidone, cariprazine, or a combination of olanzapine and fluoxetine. In yet another embodiment, the antipsychotic agent is a combination of brexpiprazole and sertraline.

### BRIEF DESCRIPTION OF THE DRAWINGS

FIG. 1 is a diagram showing the design of the phase 1 three-way, crossover study described in Example 1.

FIG. 2A is a bar graph showing the placebo-adjusted effect size of the decrease in EEG theta relative power at the Fz electrode after administration of 0.5 mg or 1.5 mg tilivapram. The reduction in theta power is denoted by a more negative effect size.

FIG. 2B are EEG power spectrum density (PSD) plots at the Fz electrode after administration of 0.5 mg or 1.5 mg tilivapram or placebo.

FIG. 3A is a bar graph showing the placebo-adjusted effect size of the increase in in EEG theta intertrial coherence (ITC) at the Cz electrode after administration of 0.5 mg or 1.5 mg tilivapram. The increase in theta ITC is denoted by a more positive effect size.

FIG. 3B are plots of intertrial coherence at different points before and after onset of an auditory stimulus (at 0 ms) at the Cz electrode after administration of 0.5 mg or 1.5 mg tilivapram or placebo.

FIG. 4A is a bar graph showing the placebo-adjusted effect size of the increase in gamma-band phase locking after auditory stimulus onset at the Pz electrode as measured by EEG after administration of 0.5 mg or 1.5 mg tilivapram.

FIG. 4B are plots of phase locking factor at different points before and after onset of an auditory stimulus (at 0 ms) at the Pz electrode after administration of 0.5 mg or 1.5 mg tilivapram or placebo.

FIG. 5A is a bar graph showing the placebo-adjusted effect size of the increase in the mismatch negativity as measured by an oddball task with EEG at electrode Fz after administration of 0.5 mg or 1.5 mg of tilivapram. A larger mismatch negativity is denoted by a more negative effect size as it is a negative-going event related potential.

FIG. 5B is a plot of the mismatch negativity event related potential at the Fz electrode after administration of 0.5 mg or 1.5 mg tilivapram or placebo. The mismatch negativity is a potential peaking around 150-200 ms.

FIG. 6A is a bar graph showing the placebo-adjusted effect size of the increase in processing speed after administration of 0.5 mg or 1.5 mg tilivapram.

FIG. 6B is a bar graph showing the placebo adjusted effect size of the decrease in finger tapping task latency after administration of 0.5 mg or 1.5 mg tilivapram. A more negative effect size denotes reduced latency (i.e. faster finger tapping).

FIG. 7 is a table of the frequency of various adverse events after administration of 0.5 mg or 1.5 mg tilivapram or placebo. TEAE=Treatment-Emergent Adverse Events.

FIGS. 8A (linear scale) and 8B (semi-log scale) show the mean plasma concentration of tilivapram versus time after administration with an oral solution or transdermal patch as described in Example 2.

FIGS. 9A (linear scale) and 9B (semi-log scale) show the mean plasma concentration of tilivapram versus time after administration with an oral solution or transdermal patch for the 24 hours relevant to that dosing period (i.e. 0-24 hours for PID1 and P2D1 and 24-48 hr for P2D2) as described in Example 2.

### DETAILED DESCRIPTION OF THE INVENTION

Unless specifically stated or obvious from context, as used herein, the term “or” is understood to be inclusive.

As used herein, the phrase “tilivapram, zatolmilast, roflumilast, or a pharmaceutically acceptable salt thereof” refers to “tilivapram or a pharmaceutically acceptable salt thereof,” “zatolmilast or a pharmaceutically acceptable salt thereof,” and “roflumilast, or a pharmaceutically acceptable salt thereof.”

Unless specifically stated or obvious from context, as used herein, the terms “a”, “an”, and “the” are understood to be singular or plural.

Ranges provided herein are understood to be shorthand for all of the values within the range.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Unless indicated otherwise, the term “tilivapram” (AVE8112) refers to 4-(cyclopropylmethoxy)-N-(3,5-dichloro-1-oxidopyridin-4-yl)-5-methoxypyridine-2-carboxamide (otherwise referred to as 4-(cyclopropylmethoxy)-N-(3,5-dichloro-1-oxido-4-pyridyl)-5-methoxypyridine-2-carboxamide), which has the structure:

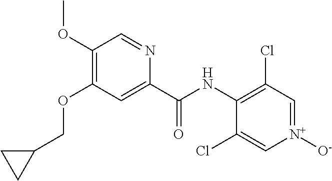 [SMILES: COc1cnc(C(=O)Nc2c(Cl)c[n+]([O-])cc2Cl)cc1OCC1CC1]

Tilivapram and pharmaceutically acceptable salts thereof can be prepared as described in U.S. Pat. No. 9,611,250 and International Publication No. WO 95/04045, each of which is incorporated by reference in its entirety.

Pharmaceutically acceptable salts include, but are not limited to, those derived from the following acids as well as acid addition salts: mineral acids such as hydrochloric acid, sulfuric acid, phosphoric acid and sulfamic acid; and organic acids such as acetic acid, citric acid, lactic acid, tartaric acid, malonic acid, methanesufonic acid, ethanesulfonic acid, benzenesulfonic acid, p-toluenesulfonic acid, cyclohexylsulfamic acid, and quinic acid. The corresponding acid addition salts comprise the following: hydrohalides, such as hydrochloride and hydrobromide, sulfate, phosphate, nitrate, sulfamate, acetate, citrate, lactate, tartrate, malonate, oxalate, salicylate, propionate, succinate, fumarate, maleate, methylene-bis-B-hydroxynaphthoates, gentisates, mesylates, isethionates and di-p-toluoyltartratesmethanesulfonate, ethanesulfonate, benzenesulfonate, p-toluenesulfonate, cyclohexylsulfamate and quinate, respectively.

The term “zatolmilast” (BPN14770) refers to 2-(4-((2-(3-chlorophenyl)-6-(trifluoromethyl)pyridin-4-yl)methyl)phenyl)-1-(I1-oxidaneyl)ethan-1-one (or 2-[4-[[2-(3-chlorophenyl)-6-(trifluoromethyl)pyridin-4-yl]methyl]phenyl]acetic acid), which has the structure shown below and is described in U.S. Pat. No. 10,093,686 (see Example 142), which is hereby incorporated by reference.

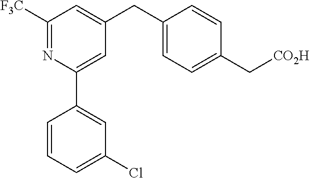 [SMILES: O=C(O)Cc1ccc(Cc2cc(-c3cccc(Cl)c3)nc(C(F)(F)F)c2)cc1]

Roflumilast has the chemical name N-(3,5-dichloropyridin-4-yl)-3-cyclopropylmethoxy-4-difluoromethoxy-benzamide. The roflumilast can be in the form of roflumilast free base, an N-oxide of roflumilast, or a pharmaceutically acceptable salt thereof. Exemplary salts of roflumilast are salt described in U.S. Patent Publication No. 2006/0084684, the entire disclosure of which is incorporated herein by reference.

The terms “treat,” “treatment,” and “treating” in the context of the administration of a therapy to a patient refers to the reduction or inhibition of the progression and/or duration of a disease or condition, the reduction or amelioration of the severity of a disease or condition, and/or the amelioration of one or more symptoms thereof resulting from the administration of one or more therapies.

The term “impaired cognition” refers to a subject having cognition, as measured by one or more tests, below that of the 50^{th} percentile, or a lower cutoff, of healthy subjects of similar demographics, such as based on similarity in age to patients (i.e., z-score<0). The z-score is an example of a standardized score that can be used to characterize subjects as “impaired”. The z-score reflects a transformation of cognitive performance relative to a healthy subject distribution, which may account for factors such as age, education and gender in that transformation. A z score below zero indicates performance for that subject that is below the 50^{th} percentile of similar healthy subjects, while a z score above zero indicates performance that is above the 50^{th} percentile of similar healthy subjects. For example, the subject may have a cognition score below the 50^{th} percentile of a similar healthy subject with a z-score less than zero, less than z=−0.25, z=−0.5, z=−0.75, z=−1, or z=−2 (e.g., with a z-score of from about −0.5 or −0.75 to about −1 or about −2, or a z-score of from about −0.75 or −1 to about −2). In one embodiment, a patient is considered to have impaired cognition when the z-score is less than −0.2, −0.25, −0.3, −0.35, −0.4, −0.45, −0.5, −0.55, −0.6, −0.65, −0.7, −0.75, −0.8, −0.85, −0.9, −0.95, or −1.0. In another embodiment, a patient is considered to have impaired cognition when the z-score is less than −1.2, −1.25, −1.3, −1.35, −1.4, −1.45, −1.5, −1.55, −1.6, −1.65, −1.7, −1.75, −1.8, −1.85, −1.9, −1.95, or −2.0. In one embodiment, impaired cognition based on a global cognition composite score. Global cognition, which is measured as a composite of multiple individual cognitive measures, is a common clinical measure of the severity of cognitive impairment associated with schizophrenia.

The term “impaired memory” refers to a subject having memory, as measured by one or more tests, below that of the 50^{th} percentile, or a lower cutoff, of healthy subjects of similar demographics, such as based on similarity in age to patients (i.e., z-score<0). The z-score is an example of a standardized score that can be used to characterize subjects as “impaired”. The z-score reflects a transformation of memory performance relative to a healthy subject distribution, which may account for factors such as age, education and gender in that transformation. A z score below zero indicates performance for that subject that is below the 50^{th} percentile of similar healthy subjects, while a z score above zero indicates performance that is above the 50^{th} percentile of similar healthy subjects. For example, the subject may have a memory score below the 50^{th} percentile of a similar healthy subject with a z-score less than zero, less than z=−0.25, z=−0.5, z=−0.75, z=−1, or z=−2 (e.g., with a z-score of from about −0.5 or −0.75 to about −1 or about −2, or a z-score of from about −0.75 or −1 to about −2). In one embodiment, a patient is considered to have impaired memory when the z-score is less than −0.2, −0.25, −0.3, −0.35, −0.4, −0.45, −0.5, −0.55, −0.6, −0.65, −0.7, −0.75, −0.8, −0.85, −0.9, −0.95, or −1.0. In another embodiment, a patient is considered to have impaired memory when the z-score is less than −1.2, −1.25, −1.3, −1.35, −1.4, −1.45, −1.5, −1.55, −1.6, −1.65, −1.7, −1.75, −1.8, −1.85, −1.9, −1.95, or −2.0. In one embodiment, impaired memory is assessed (partly or wholly) based on recall index.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, delay, inhibition, suppression, or reduction of a symptom or symptoms of a disease or disorder, which could also be referred to as a “therapeutically effective amount.” A “reduction” of a symptom or symptoms (and grammatical equivalents of this phrase) means decreasing of the severity or frequency of the symptom(s), or elimination of the symptom(s). An “effective amount” of a drug can be an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Dosages may be varied depending upon the requirements of the patient and the compound being employed. The dose administered to a patient, in the context of the present disclosure, should be sufficient to effect a beneficial therapeutic response in the patient over time. The size of the dose may also be determined by the existence, nature, and extent of any adverse side-effects. Determination of the proper dosage for a particular situation is within the skill of the practitioner.

As used herein, the terms “subject,” “participant,” and “patient” are used interchangeably and refer to a human patient unless indicated otherwise.

The term “transdermal administration” refers to a route of administration in which the pharmaceutical dosage form is taken up through the skin. Similarly, a “transdermal delivery device” is intended to mean any apparatus or system that administers a drug to be taken up through the skin.

Transdermal delivery devices are known in the art. Thus, any device suitable for delivery of drug across the skin of a patient may be used. Devices known in the art include reservoir type devices involving membranes that control the rate of drug release to the skin and devices where the drug is dispersed or dissolved in a matrix such as a pressure sensitive adhesive. Transdermal delivery devices may be made in the form of an article such as a tape, a patch, a sheet, a dressing or any other form known in the art. Generally, the device may in the form of a patch of a size suitable to deliver a preselected amount of drug through the skin. In one embodiment, the device has a surface area of about 5 cm² to about 100 cm², such as about 10 cm² to about 40 cm².

Transdermal delivery devices typically involve a carrier (such as a liquid, gel, or solid matrix, or a pressure sensitive adhesive) into which the tilivapram, zatolmilast, roflumilast, or a pharmaceutically acceptable salt thereof is incorporated. In one embodiment, transdermal administration is by a sustained release or extended release transdermal formulation and/or device. In some embodiments, sustained release transdermal administration of the tilivapram, zatolmilast, roflumilast, or pharmaceutically acceptable salt thereof includes multi-day delivery of a therapeutically effective amount of the tilivapram, zatolmilast or roflumilast that is applied to the skin of a subject. By multi-day delivery is meant that the transdermal composition is formulated to provide a therapeutically effective amount to a subject when the transdermal delivery device is applied to the skin of a subject for a period of time that is 1 day or longer, such as 2 days or longer, such as 4 days or longer, such as 7 days or longer, such as 14 days and including 30 days or longer. In certain embodiments, transdermal delivery devices provide a therapeutically effective amount of the tilivapram, zatolmilast, or roflumilast to a subject for a period of 10 days or longer. For multi-day delivery, an upper limit period of time is, in some instances, 30 days or shorter, such as 28 days or shorter, such as 21 days or shorter, such as 14 days or shorter, such as 7 days or shorter and including 3 days or shorter. In certain embodiments, multi-day transdermal delivery ranges such as from 2 days to 30 days, such as from 3 days to 28 days, such as from 4 days to 21 days, such as from 5 days to 14 days and including from 6 days to 10 days.

Methods for making formulations suitable for transdermal administration are known in the art, such as in Remington: The Science and Practice of Pharmacy, 23^{rd} Ed. (Academic Press), 2020.

Suitable doses of tilivapram or a pharmaceutically acceptable salt thereof for transdermal administration include from about 0.01 to about 50 mg/kg body weight per day, such as about 0.1 to about 40 mg/kg body weight per day or about 0.5 to about 10 mg/kg body weight per day. In one embodiment, about 1 mg to about 700 mg of tilivapram or a pharmaceutically acceptable salt thereof is transdermally administered daily.

Suitable doses of zatolmilast or a pharmaceutically acceptable salt thereof for transdermal administration include from about 0.01 to about 100 mg/kg body weight per day, such as about 0.1 to about 70 mg/kg body weight per day or about 0.5 to about 10 mg/kg body weight per day. In one embodiment, about 10 to about 700 mg of zatolmilast or a pharmaceutically acceptable salt thereof are transdermally administered daily.

Suitable doses of roflumilast or a pharmaceutically acceptable salt thereof for transdermal administration include from about 0.01 to about 100 mg/kg body weight per day, such as about 0.1 to about 70 mg/kg body weight per day or about 0.5 to about 10 mg/kg body weight per day. In one embodiment, about 0.5 to about 700 mg (e.g., about 1 to about 10 mg or about 1 to about 5 mg) of roflumilast or a pharmaceutically acceptable salt thereof are transdermally administered daily.

In one embodiment, the method comprises transdermally administering from about 10 to about 40 mg of tilivapram or a pharmaceutically acceptable salt thereof daily, such as from about 15 to about 30 mg or from about 18 to about 27 mg of tilivapram or a pharmaceutically acceptable salt thereof daily. In another embodiment, about 18 mg of tilivapram free base is administered daily. In yet another embodiment, about 27 mg of tilivapram free base is administered daily.

PDE4 inhibitors such as tilivapram have well-known class-related dose-limiting side effects of nausea and vomiting at higher doses. This is thought to be due to its action in the brainstem at the area postrema (also known as the chemoreceptor trigger zone). In an immediate release formulation, the maximum drug dose is achieved at Cₘₐₓ (the highest concentration of a drug in the blood, cerebrospinal fluid, or target organ after a dose is given). In contrast, the effective dose of a drug relates more so to the AUC (area under the curve), and more meaningfully to the time over which a behaviorally relevant blood level of the drug is achieved. As such, in the service of maximizing the time of exposure to the behaviorally relevant blood level, giving a patient a higher dose of the drug will end up also achieving a higher Cₘₐₓ, and thereby a higher likelihood of dose-related nausea/vomiting and related adverse events such as dizziness or lightheadedness. By giving the drug transdermally, the time at the behaviorally relevant blood level can be maximized while simultaneously minimizing Cₘₐₓ, and thereby diminishing dose- limiting side effects.

Transdermal patches for long term use (such as weekly, biweekly, or monthly use) can also provide improved patient compliance and consistent and continuous treatment. An immediate release dosage form would have to be administered at a significantly greater frequency than that provided transdermally over a sustained period in order to achieve the same time during which a pharmacodynamically relevant concentration of the drug remains in the blood. Such an immediate release dosage form would also result in greater side effects (and possibly render the dosage form intolerable) due to the higher Cₘₐₓ which is achieved and/or the frequency with which Cₘₐₓ spikes are induced due to taking an individual oral immediate release dosage.

In one embodiment of any of the methods described herein, the tilivapram, zatolmilast, roflumilast, or a pharmaceutically acceptable salt thereof is administered via a transdermal patch. The transdermal patch may also administer the one or more (e.g., one or two) antipsychotic agents. In one embodiment, each transdermal patch comprises from about 4 to about 30 mg of tilivapram or a pharmaceutically acceptable salt thereof, such as from about 18 to about 27 mg of tilivapram or a pharmaceutically acceptable salt thereof or about 4.5, 9, 13.5, 18, 22.5, or 27 mg of tilivapram or a pharmaceutically acceptable salt thereof. For example, each transdermal patch may comprise from about 4 to about 30 mg of tilivapram free base, such as from about 18 to about 27 mg of tilivapram free base or about 4.5, 9, 13.5, 18, 22.5, or 27 mg of tilivapram free base.

In one embodiment of any of the methods described herein, the concentration of tilivapram, zatolmilast, roflumilast, or a pharmaceutically acceptable salt thereof at 2 hours after initiating administration with the drug for the first time is less than about 20%, such as less than about 15%, less than about 10%, or less than about 5% of Cₘₐₓ for the first 24 hours (from the time administration is initiated). In one embodiment of any of the methods described herein, the concentration of tilivapram, zatolmilast, roflumilast, or a pharmaceutically acceptable salt thereof at 2 hours after initiating administration with the drug for the first time is less than about 5% of Cₘₐₓ for the first 24 hours (from the time administration is initiated). In one embodiment, after 1-4 hours from initiating administration with the drug for the first time, the plasma concentration of tilivapram, zatolmilast, or roflumilast rises at a zero order rate over the next 10 to 24 hours. Without being bound by any particular theory, the inventors theorize that the slow rise in the blood concentration of the drug minimizes adverse effects.

In one embodiment of any of the methods described herein, the tilivapram, zatolmilast, roflumilast, or a pharmaceutically acceptable salt thereof is transdermally administered over at least about 12 hours, at least about 18 hours, at least about 20 hours, or at least about 24 hours.

In one embodiment of any of the methods described herein, the tilivapram, zatolmilast, roflumilast, or a pharmaceutically acceptable salt thereof is transdermally administered with a transdermal delivery device (such as a patch). In one embodiment of any of the methods described herein, the patch is replaced daily. In another embodiment of any of the methods described herein, the patch is replaced once every two days, once every three days, once every four days, once every five days, once every six days, or once a week.

At steady state, the transdermal delivery device (even when replaced with a new device) provides low fluctuation and swing of tilivapram, zatolmilast, or roflumilast blood or plasma levels. In one embodiment, the fluctuation (for example, over a 24 hour period at steady state) is less than 100%, 90%, 80%, 70%, 60%, 50% or 40%. In another embodiment, the swing (for example, over a 24 hour period at steady state) is less than 120%, 110%, 100%, 90%, 80%, 70%, 60%, 50%, or 40%. Swing denotes (Cₘₐₓ-Cₘᵢₙ)/Cₘᵢₙ and fluctuation denotes (Cₘₐₓ-Cₘᵢₙ)/C_{average}.

The transdermal delivery device, such as a transdermal patch, may remain on the patient for extended periods of time to provide continuous delivery of the tilivapram. For instance, the patch may be work for 1 week or 2 weeks (e.g., a new patch may replace a currently worn patch once a week or biweekly).

Alternatively, administration may be once a day (q.d.) or at less frequent intervals such as once every other day (q.a.d.), once every third day, twice a week (bis in 7 d.), once a week (QWK), or once every other week. In another embodiment, administration can be twice daily (b.i.d.).

In one embodiment, administration is by the oral route, such as once a day (q.d.), twice daily (b.i.d.), three times daily (t.i.d.), or four times daily (q.i.d.). In another embodiment, oral administration is by a sustained release or extended release oral formulation.

In one embodiment of any of the methods described herein, the one or more antipsychotic agents are selected from olanzapine, risperidone, quetiapine, sertindole, amisulpride, aripiprazole, asenapine, blonanserin, bifeprunox, cariprazine, clotiapine, iloperidone, lurasidone, mosapramine, melperone, paliperidone, perospirone, pimavanserin, remoxipride, sulpiride, ziprasidone, zotepine, perphenazine, thioridazine, chlorpromazine, brexpiprazole, lumateperone (e.g., lumateperone tosylate), fluphenazine, haloperidol, loxapine, prochlorperazine, flupentixol, levomepromazine, periciazine, pimozide, promazine, trifluperazine, zuclopenthixol, molindone, thiothixene, thoridazine, trifluoperazine, pharmaceutically acceptable salts thereof, and any combination of any of the foregoing.

In one embodiment of any of the methods described herein, one antipsychotic agent is administered to the patient. In one embodiment of any of the methods described herein, two antipsychotic agents are administered to the patient.

In one embodiment of any of the methods described herein, the one or more antipsychotic agents are administered orally.

In one embodiment of any of the methods described herein, the patient is administered one or more dosage forms, where each dosage form comprises (a) tilivapram, zatolmilast, roflumilast, or a pharmaceutically acceptable salt thereof and (b) one or more antipsychotic agents. In one embodiment, the one or more dosage forms are administered transdermally.

### EXAMPLE 1

### Phase 1 Dose Response Study

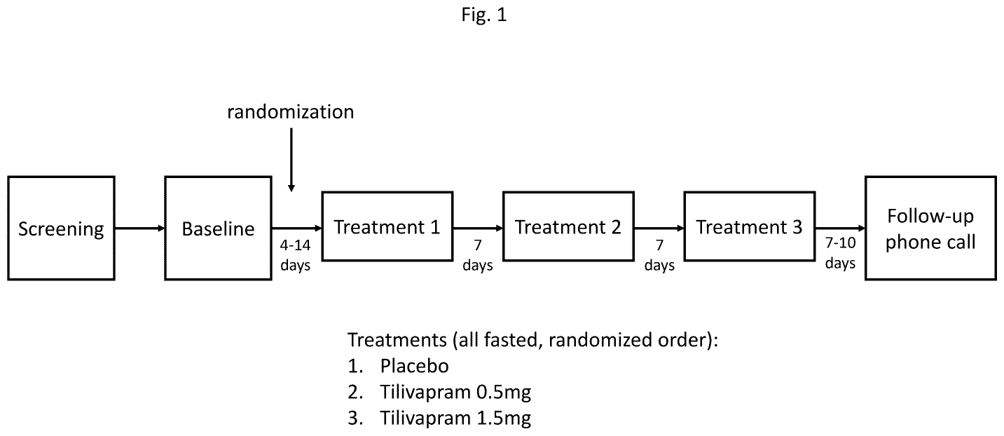

A phase 1 three-way, crossover study was performed as shown in FIG. 1 on healthy patients aged 40 to 65 years old to determine dose and pharmacodynamic outcomes. Patients in the fasted state were orally administered 0.5 or 1.5 mg tilivapram or placebo in the form of an immediate release suspension. Outcomes were measured with a computerized neurocognitive test battery, resting 19 channel EEG, and cognitive task ERPs (event-related potentials). 44 patients were dosed (and used for AE analyses), of which 40 received all three doses (and used for analysis of cognitive, EEG and ERP outcomes).

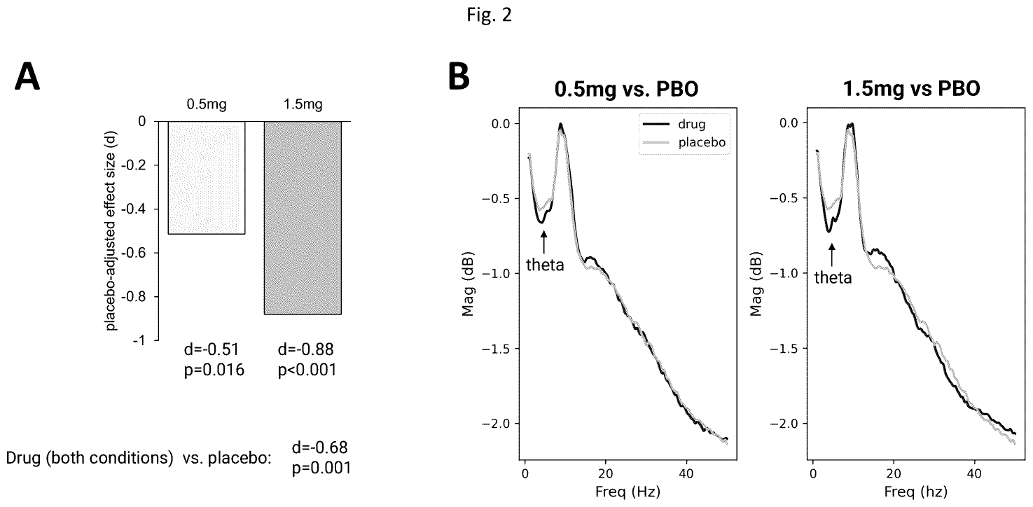

Theta (4-8 Hz) is an EEG rhythm involving connections between the hippocampus and prefrontal cortex. Frontal midline resting theta power is elevated across a range of cognitive disorders, such as schizophrenia, ADHD, Parkinson's disease, Alzheimer's disease and mild cognitive impairment (MCI) (see Newson et al., Front Hum Neurosci., 2019, 12:521 (PMID 30687041)). Increased theta power correlates with poorer cognitive task performance. Tilivapram at 0.5 and 1.5 mg decreased theta relative power at the Fz electrode, as shown by FIGS. 2A and 2B. Tilivapram has a wide therapeutic window as there were significant decreases in theta power at both the 0.5 mg and 1.5 mg doses.

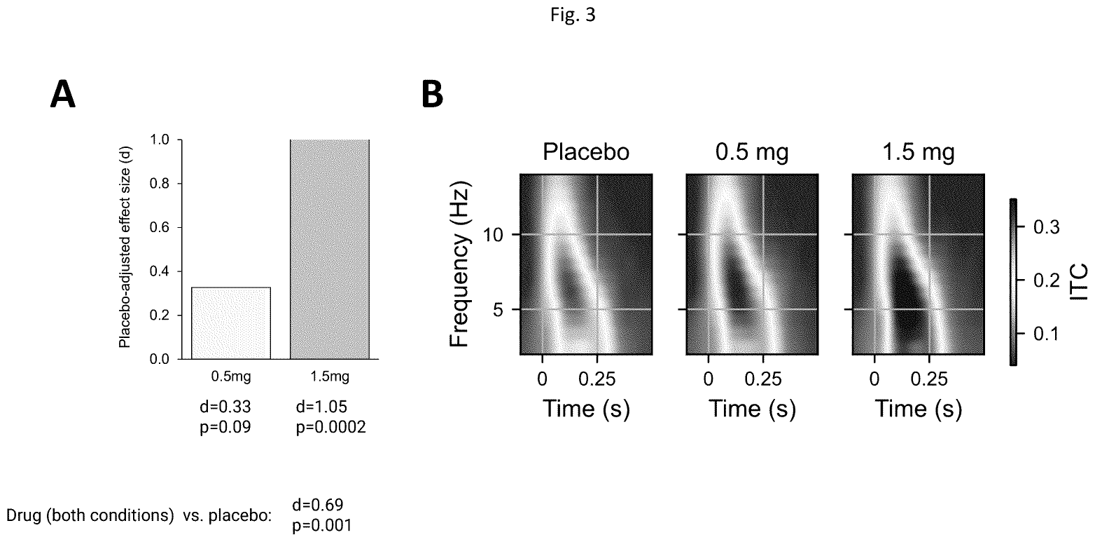

Stimulus-induced theta response, such as measured by inter-trial coherence, phase locking factor or event-related spectral perturbation, reflect the brain response during stimulus processing, is reduced in patients with schizophrenia, and the degree of reduction is correlated with the degree of cognitive impairment in these patients (Lee at al., Mol Psychiatry, 2017, 22 (11): 1585-1593, PMID 28167837; Martínez et al., Front Hum Neurosci., 2015, 9:371, PMID 26190988; Xiong et al.,Front Hum Neurosci., 2019, 13:37, PMID 30894804; Hua et al., Biol Psychiatry Cogn Neurosci Neuroimaging., 2023, 8 (12): 1186-1196, PMID: 36931469; Hochberger et al., Int. J. Psychophysiology, 2019, 145:23-29, PMID: 30586570; Wolff et al., Cerebral Cortex, 2022, 32:3441-3456: PMID: 34875019). Tilivapram at 0.5 mg and 1.5 mg increased theta intertrial coherence at the Cz electrode, as shown by FIGS. 3A and 3B.

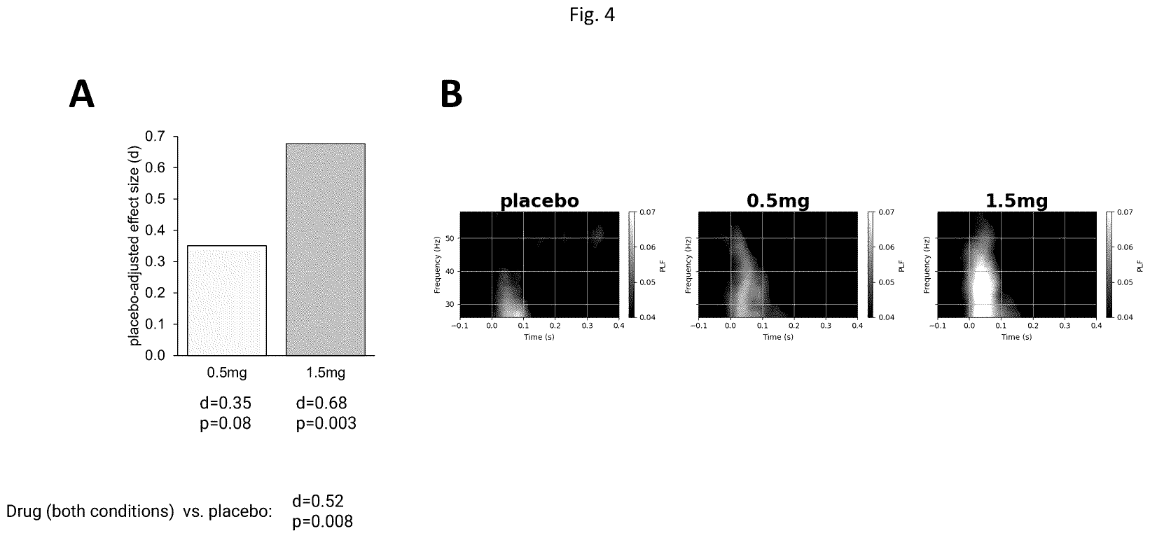

Evoked sensory gamma-based responses represent both basic sensory processing as well as higher cognitive functions, such as attention and memory. The onset of a stimulus triggers precisely timed local processing, evident as an increase in gamma-band intertrial phase locking. Gamma-band phase locking at ˜50 ms after stimulus onset is reduced in schizophrenia and other psychotic spectrum disorders such as bipolar disorder (see Roach et al., Schizophr Bull., 34 (5): 907-26, 2008 (PMID 18684772); Javitt et al., Neuropsychopharmacology, 45 (9): 1411-1422, 2020 (PMID 32375159)). Tilivapram at 0.5 and 1.5 mg increased gamma-band phase locking approximately 50 ms after onset of a tone auditory stimulus (FIGS. 4A and 4B).

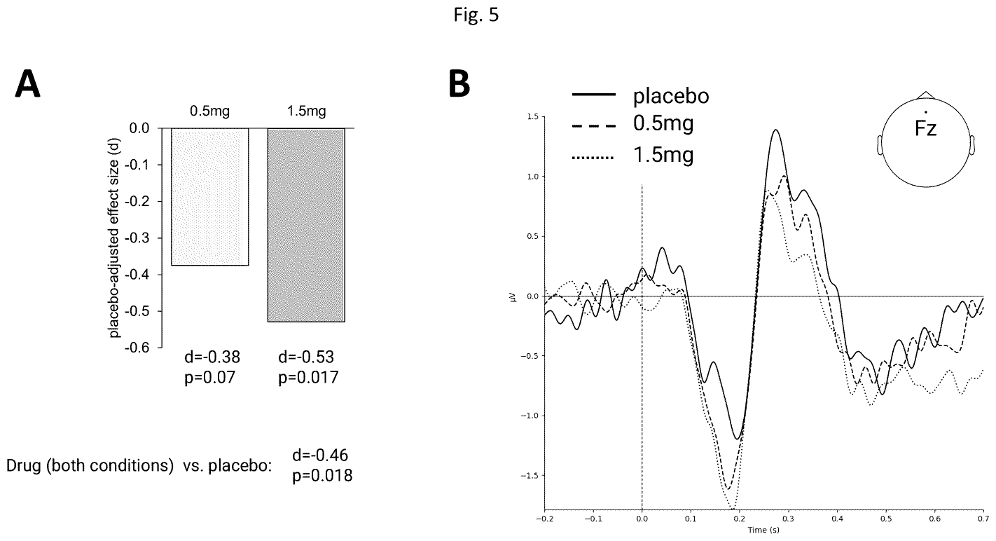

Mismatch negativity (MMN) is a well-defined event-related potential biomarker indicative of information filtering and low-level attention, which is reduced (i.e. less negative potential) in schizophrenia and related psychotic spectrum conditions (e.g., bipolar disorder) (see Kim et al., Front Psychiatry, 11:795, 2020 (PMID 32848953); Todd et al., Front Psychiatry, 4:171, 2013 (PMID 24391602); Wang et al., Transl Psychiatry, 12 (1): 100, 2022 (PMID 35277479)). A reduction in MMN correlates with poorer cognitive task performance in these patients. Tilivapram at 0.5 and 1.5 mg increased MMN (i.e. more negative potential) at the Fz electrode as shown in FIGS. 5A and 5B.

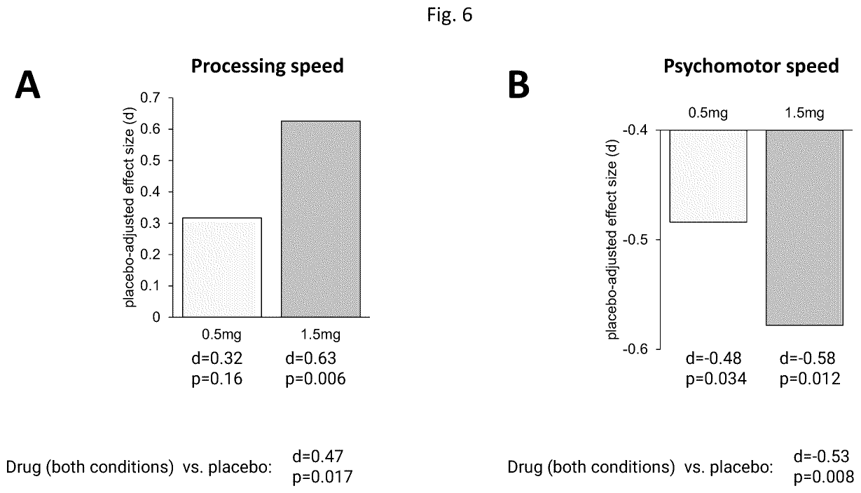

Tilivapram at 0.5 and 1.5 mg also improved processing speed, which is a composite z-score comprised of age and gender-normed z-scores from the simple reaction time, choice reaction time and Eriksen flanker tasks (FIG. 6A). Tilivapram at 0.5 and 1.5 mg also improved psychomotor speed, evident as reduced latency in a finger tapping task (FIG. 6B). Tilivapram has a wide therapeutic window in the finger tapping task as there were significant decreases in latency at both the 0.5 mg and 1.5 mg doses.

In summary, treatment with tilivapram results in decreased theta power, increased theta intertrial coherence, increased gamma-band phase locking, increased mismatch negativity, and increased both processing speed and psychomotor speed. These are consistent with pro- cognitive effects.

The effect of tilivapram versus placebo on cognition was examined between subgroups of the study sample differing in their global cognitive composite score at baseline. Restricting to participants with below-average global cognition at baseline resulted in increased positive effect sizes of tilivapram on global cognition and memory. For example, global cognition showed a small and non-significant difference between the 0.5 mg dose and placebo in the full sample (d=0.274, p=0.223). However, examining only participants with baseline global cognition below healthy norms (z<−0.5), the effect of 0.5 mg tilivapram relative to placebo increased to d=0.861, p=0.034. Similarly, memory showed a small and non-significant difference between 0.5 mg and placebo in the full sample (d=0.207, p=0.359). Examining participants with global cognition of z<−0.5, the effect on memory becomes larger and statistically significant (d=0.797, p=0.048).

Global cognition, which is measured as a composite of multiple individual cognitive measures, is a common clinical measure of the severity of cognitive impairment associated with schizophrenia. One important component of the cognitive impairment in these patients is memory.

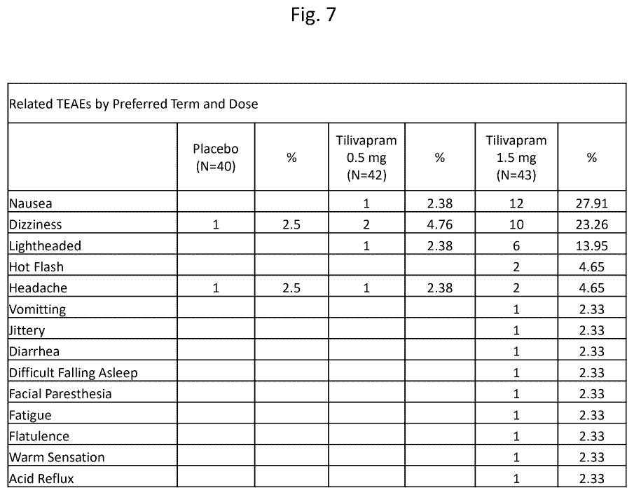

Adverse events increased substantially from the 0.5 mg dose (which was similar to placebo) to the 1.5 mg dose (FIG. 7). The adverse events were primarily nausea, as expected given the well-known dose-limiting effects of PDE4 inhibitors on nausea. Approximately 28% of people experienced nausea at the 1.5 mg dose, while only 2% did so at the 0.5 mg dose. Dizziness and lightheadedness was also substantially increased in a dose-related manner. Only one patient discontinued treatment due to an adverse event at the higher dose (1.5 mg) of tilivapram.

### EXAMPLE 2

### Clinical Study

A phase I, open-label, fixed period, 2-way cross-over study was performed to establish the pharmacokinetics and safety of a transdermal delivery system containing tilivapram. In Period 1 Day 1 (P1D1), participants were dosed with 1.0 mg of tilivapram in a liquid oral solution in a fasted state. After a 7-day washout period, participants were dosed with the first set of 4 patches applied by study staff on Period 2 Day 1 (P2D1), which were then removed after 24 hours and 4 new patches applied for an additional 24 hours on Period 2 Day 2 (P2D2) for a total of 48 hours. All but one of the 15 subjects completed the study. One subject withdrew after receiving the oral solution.

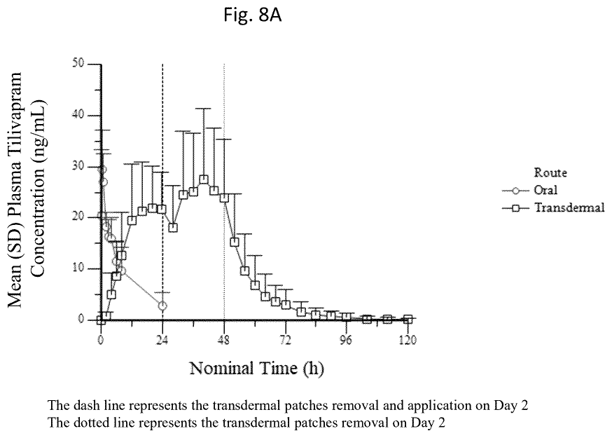

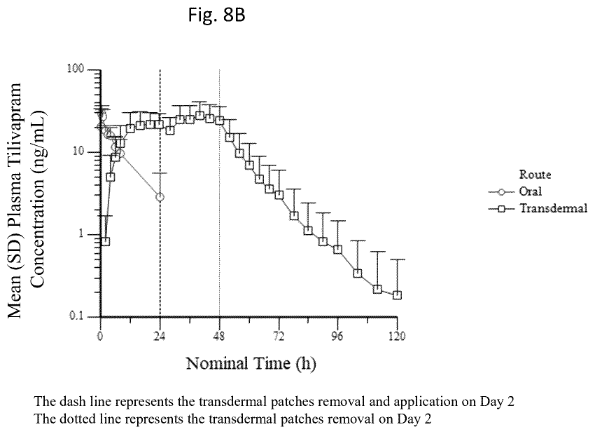

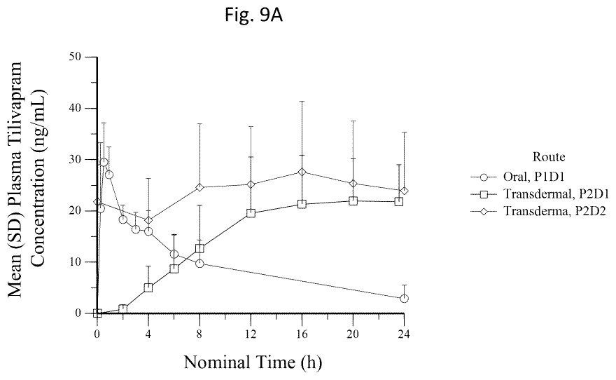

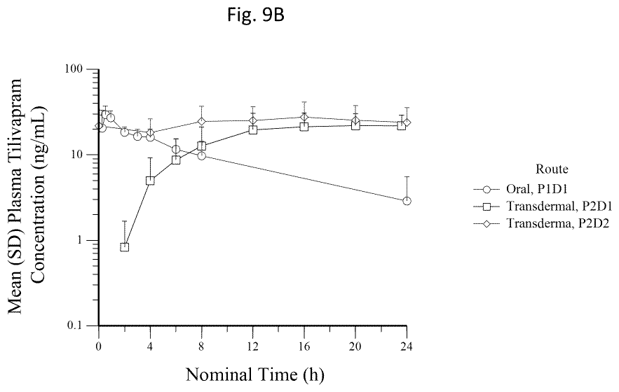

The mean plasma concentration of tilivapram administered orally and transdermally versus time is shown in a linear scale in FIG. 8A and a semi-log scale in FIG. 8B, across the entire period of the study. The mean plasma concentration of tilivapram administered orally and transdermally versus time for the 24 hours relevant to that dosing period (i.e. 0-24 hours for P1D1 and P2D1 and 24-48 hr for P2D2) is shown in a linear scale in FIG. 9A and a semi-log scale in FIG. 9B.

The related adverse events resulting from the oral solution and transdermal patches are provided in the table below.

| | | | |
|---|---|---|---|
| MedDRA System | | | |
| Organ Class | | Transdermal | |
| Preferred Term | Oral Solution | Formulation | |
| Relationship | (N = 15) | (N = 14) | Overall (N = 15) |

Number of participants with at least 1 TEAE, n (%)

| | | |
|---|---|---|
| 9 (60.0) | 8 (57.1) | 11 (73.3) |

Number of TEAEs

| | | |
|---|---|---|
| 15 | 13 | 28 |

Nervous system disorders, n (%)

| | | |
|---|---|---|
| 6 (40.0) | 6 (42.9) | 8 (53.3) |

Dizziness, n (%)

| | | |
|---|---|---|
| 6 (40.0) | 1 (7.1) | 6 (40.0) |

Headache, n (%)

| | | |
|---|---|---|
| 2 (13.3) | 5 (35.7) | 6 (40.0) |

Neuralgia, n (%)

| | | |
|---|---|---|
| 0 | 0 | 0 |

Gastrointestinal disorders, n (%)

| | | |
|---|---|---|
| 3 (20.0) | 0 | 3 (20.0) |

Nausea, n (%)

| | | |
|---|---|---|
| 3 (20.0) | 0 | 3 (20.0) |

Diarrhea, n (%)

| | | |
|---|---|---|
| 1 (6.7) | 0 | 1 (6.7) |

Abdominal pain, n (%)

| | | |
|---|---|---|
| 0 | 0 | 0 |

Dyspepsia, n (%)

| | | |
|---|---|---|
| 1 (6.7) | 0 | 1 (6.7) |

General disorders and administration site conditions, n (%)

| | | |
|---|---|---|
| 1 (6.7) | 2 (14.3) | 2 (13.3) |

Administration site pruritus, n (%)

| | | |
|---|---|---|
| 0 | 2 (14.3) | 2 (13.3) |

Administration site erythema, n (%)

| | | |
|---|---|---|
| 0 | 0 | 0 |

Administration site pain, n (%)

| | | |
|---|---|---|
| 0 | 0 | 0 |

Asthenia, n (%)

| | | |
|---|---|---|
| 1 (6.7) | 0 | 1 (6.7) |

Fatigue, n (%)

| | | |
|---|---|---|
| 0 | 0 | 0 |

Psychiatric disorders, n (%)

| | | |
|---|---|---|
| 0 | 0 | 0 |

Insomnia, n (%)

| | | |
|---|---|---|
| 0 | 0 | 0 |

Irritability, n (%)

| | | |
|---|---|---|
| 0 | 0 | 0 |

Ear and labyrinth disorders, n (%)

| | | |
|---|---|---|
| 1 (6.7) | 0 | 1 (6.7) |

Vertigo, n (%)

| | | |
|---|---|---|
| 1 (6.7) | 0 | 1 (6.7) |

Musculoskeletal and connective tissue disorders, n (%)

| | | |
|---|---|---|
| 0 | 0 | 0 |

Arthralgia, n (%)

| | | |
|---|---|---|
| 0 | 0 | 0 |

Skin and subcutaneous tissue disorders, n (%)

| | | |
|---|---|---|
| 0 | 0 | 0 |

Dermatitis acneiform, n (%)

| | | |
|---|---|---|
| 0 | 0 | 0 |

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A method of treating a neuropsychiatric disorder in a patient in need thereof comprising administering to the patient an effective amount of tilivapram, zatolmilast, roflumilast, or a pharmaceutically acceptable salt thereof.

2. A method of treating negative symptoms of schizophrenia in a patient in need thereof comprising administering to the patient an effective amount of tilivapram, zatolmilast, roflumilast, or a pharmaceutically acceptable salt thereof.

3-5. (canceled)

6. A method of treating a neuropsychiatric disorder in a patient in need thereof comprising administering to the patient an effective amount of (a) tilivapram, zatolmilast, roflumilast, or a pharmaceutically acceptable salt thereof and (b) one or more antipsychotic agents.

7. The method of claim 2, further comprising administering one or more antipsychotic agents.

8-10. (canceled)

11. The method of claim 6, wherein the one or more antipsychotic agents are selected from olanzapine, risperidone, quetiapine, sertindole, amisulpride, aripiprazole, asenapine, blonanserin, bifeprunox, cariprazine, clotiapine, iloperidone, lurasidone, mosapramine, melperone, paliperidone, perospirone, pimavanserin, remoxipride, sulpiride, ziprasidone, zotepine, perphenazine, thioridazine, chlorpromazine, brexpiprazole, lumateperone, fluphenazine, haloperidol, loxapine, prochlorperazine, flupentixol, levomepromazine, periciazine, pimozide, promazine, trifluperazine, zuclopenthixol, molindone, thiothixene, thoridazine, trifluoperazine, pharmaceutically acceptable salts thereof, and any combination of any of the foregoing.

12. The method of claim 6, wherein the one or more antipsychotic agents are administered orally.

13. The method of claim 6, wherein the patient is administered one or more dosage forms, where each dosage form comprises (a) tilivapram, zatolmilast, roflumilast, or a pharmaceutically acceptable salt thereof and (b) one or more antipsychotic agents.

14. The method of claim 13, wherein the one or more dosage forms are administered transdermally.

15. The method of claim 1, wherein the tilivapram, zatolmilast, roflumilast, or a pharmaceutically acceptable salt thereof is administered orally.

16. The method of claim 1, wherein the tilivapram, zatolmilast, roflumilast, or a pharmaceutically acceptable salt thereof is administered transdermally.

17. The method of claim 1, wherein the tilivapram or a pharmaceutically acceptable salt thereof is administered orally.

18. The method of claim 1, wherein the tilivapram or a pharmaceutically acceptable salt thereof is administered transdermally.

19. The method of claim 18, wherein the method comprises transdermally administering from about 10 to about 40 mg of tilivapram or a pharmaceutically acceptable salt thereof daily, such as from about 15 to about 30 mg or from about 18 to about 27 mg of tilivapram or a pharmaceutically acceptable salt thereof daily.

20. The method of claim 1, wherein tilivapram free base is transdermally administered to the patient.

21. The method of claim 1, wherein the tilivapram or a pharmaceutically acceptable salt thereof is administered via a transdermal patch.

22. The method of claim 21, wherein each transdermal patch comprises from about 4 to about 30 mg of tilivapram or a pharmaceutically acceptable salt thereof.

23. The method of claim 14, wherein the concentration of tilivapram, zatolmilast, roflumilast, or a pharmaceutically acceptable salt thereof at 2 hours, after initiating administration with the tilivapram, zatolmilast, or roflumilast for the first time, is less than 20%, of Cₘₐₓ for the first 24 hours (from the time administration is initiated).

24. The method of claim 14, wherein the tilivapram, zatolmilast, roflumilast, or a pharmaceutically acceptable salt thereof is transdermally administered over at least 12 hours.

25. The method of claim 14, wherein after 1-4 hours from initiating administration with the tilivapram, zatolmilast, or roflumilast for the first time, the plasma concentration of tilivapram, zatolmilast, or roflumilast rises at a zero order rate over the next 10 to 24 hours.

26. The method of claim 14, wherein at steady state, the fluctuation (for example, over a 24 hour period at steady state) of tilivapram, zatolmilast, or roflumilast blood levels is less than 100%.

27. The method of claim 14, wherein at steady state, the swing (for example, over a 24 hour period at steady state) of tilivapram, zatolmilast, or roflumilast blood levels is less than 120%.

28. The method of claim 1, wherein the neuropsychiatric disorder is Parkinson's disease.

29. The method of claim 1, wherein the neuropsychiatric disorder is cognitive impairment associated with Parkinson's disease.

30. The method of claim 1, wherein the neuropsychiatric disorder is memory impairment associated with Parkinson's disease.

31. The method of claim 1, wherein the neuropsychiatric disorder is information processing speed and/or psychomotor speed impairment associated with Parkinson's disease.

32. The method of claim 1, wherein the neuropsychiatric disorder is schizophrenia.

33. The method of claim 1, wherein the neuropsychiatric disorder is cognitive impairment associated with schizophrenia.

34. The method of claim 1, wherein the neuropsychiatric disorder is memory impairment associated with schizophrenia.

35. The method of claim 1, wherein the neuropsychiatric disorder is information processing speed and/or psychomotor speed impairment associated with schizophrenia.

36. The method of claim 1, wherein the neuropsychiatric disorder is mild cognitive impairment (MCI).

37. The method of claim 1, wherein the neuropsychiatric disorder is attention deficit hyperactivity disorder (ADHD).

38. The method of claim 1, wherein the neuropsychiatric disorder is a condition associated with cerebral metabolic inhibition, depression, bipolar disorder, post-traumatic stress disorder (PTSD), autism, Fragile X disorder, Angelman's syndrome, cognitive impairment, Alzheimer's disease (AD), frontotemporal dementia, vascular dementia, or dementia with Lewy bodies (DLB).

39. The method of claim 1, wherein the neuropsychiatric disorder is major depressive disorder.

40. The method of claim 1, wherein the patient has impaired cognition.

41. The method of claim 29, wherein impaired cognition is determined based on a global cognition score.

42. The method of claim 1, wherein the patient has impaired memory.
